(19) United States
(12) Patent Application Publication
(10) Pub. No.: US 2020/0100978 A1
(43) Pub. Date: Apr. 2, 2020

(54) New Crutch System

(71) Applicant: Daryl Dion Moody, Indianapolis, IN (US)

(72) Inventor: Daryl Dion Moody, Indianapolis, IN (US)

(21) Appl. No.: 16/557,568

(22) Filed: Aug. 30, 2019

Related U.S. Application Data
(60) Provisional application No. 62/738,545, filed on Sep. 28, 2018.

(51) Int. Cl. A61H 3/02; A61F 5/01

(52) U.S. Cl. CPC A61H 3/02; A61F 5/0106; A61H 2201/1676; A61H 2201/0192; A61H 2201/1664; A61F 5/0111

(57) ABSTRACT

The present invention is a combination of a telescopic walking-aid apparatus, a knee brace and a leg stabilizer. The user may secure the entire injured leg to the leg stabilizer by the plurality of straps. After the injured leg is secured to the leg stabilizer, the user may secure the knee brace to the front of the knee and provides a total protection around the entire knee. Moreover, the telescopic walking-aid apparatus allows the medical personnel or the user to adjust the length of the crutch to accommodate different stage of healing process, so the user does not need to purchase or change crutch throughout the course of healing. Finally, the replaceable foot makes the walking-aid apparatus more adaptive toward different terrains.

The current application claims a priority to the U.S. Provisional Patent application Ser. No. 62/738,545 filed on Sep. 28, 2018.

### FIELD OF THE INVENTION

The present invention generally relates to a new crutch system. More specifically, the present invention is a combination of a knee brace, an adjustable walking-aid apparatus, a leg stabilizer, and a plurality of stabilizer straps.

### BACKGROUND OF THE INVENTION

Leg injuries are often severe and take a long time to recover. Before the injured leg is fully recovered, the patient needs to use various accessories to move from place to place. However, those walking accessories require further adjustments throughout the recovery process and create difficulties and inconvenience. For example, the length of the crutch requires several adjustments before the broken leg is fully healed. This requires several hospital visits and incurs more medical bill to pay for medical personnel and new crutches. Moreover, the walking accessory provided by the medical personnel is usually not adjustable, which may create further injury during the recovery process.

The present invention solves all the above issues by providing a combination of a customizable walking-aid accessory, a knee brace, a leg stabilizer, and a plurality of stabilizer straps. Instead of replacing the cane with the crutch during multiple visits to the doctor's office, the user or medical personnel may simply adjust the customizable walking-aid accessory according to patient's height, weight, type of injury sustained, or stage of recovery. The present invention is applicable to many leg injuries, including but not limited to: sprain, strain, tear, total knee replacement, Anterior Cruciate Ligament (ACL), Medial Collateral Ligament (MCL), Posterior Cruciate Ligament (PCL), broken bone, or Achilles heel injury. The present invention is also capable to provide a solid support to the injured limb with the knee brace and/or the leg stabilizer that utilizes the plurality of stabilizer straps. Resultantly, the present invention is capable of maintaining the injured limb in a relatively stable position even during movement, and significantly reduce the recovery time.

### BRIEF DESCRIPTION OF THE DRAWINGS

FIG. 1 is a perspective view of the entire present invention.

FIG. 2 is a perspective view of the walking-aid apparatus.

FIG. 3 is a perspective view of the leg stabilizer.

FIG. 4 is a front view of the leg stabilizer, showing the plurality of medial strap openings and the plurality of lateral strap openings.

FIG. 5 is a perspective view of the leg stabilizer with the plurality of stabilizer straps, wherein the plurality of stabilizer straps is looped through the plurality of medial strap openings and the plurality of lateral strap openings.

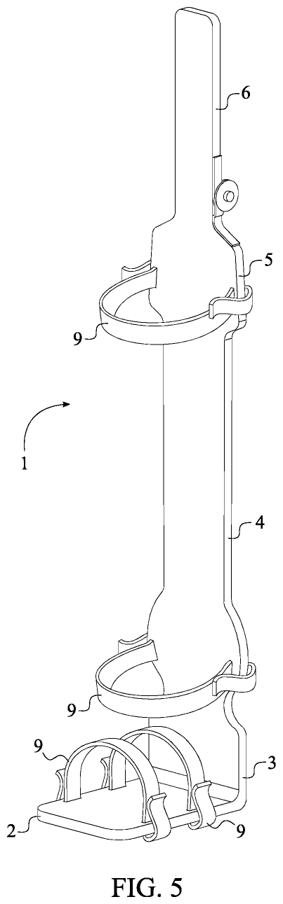

FIG. 6 is a front perspective view of the knee brace, showing the plurality of strap openings.

FIG. 7 is a back perspective view of the knee brace, wherein the plurality of brace straps is inserted through the plurality of strap openings.

FIG. 8 is a perspective exploded view of the walking-aid apparatus.

FIG. 9 is a lower perspective view of the toe body with the first support.

FIG. 10 is a lower perspective view of the toe body with the first support, the second support, and the third support.

FIG. 11 is a lower perspective view of the toe body with the first support, the second support, the third support, and the fourth support.

### DETAIL DESCRIPTIONS OF THE INVENTION

All illustrations of the drawings are for the purpose of describing selected versions of the present invention and are not intended to limit the scope of the present invention.

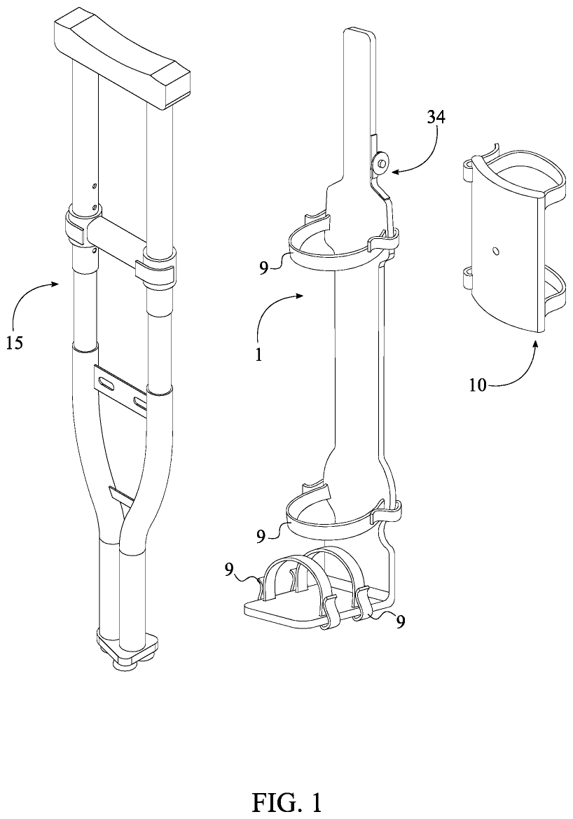

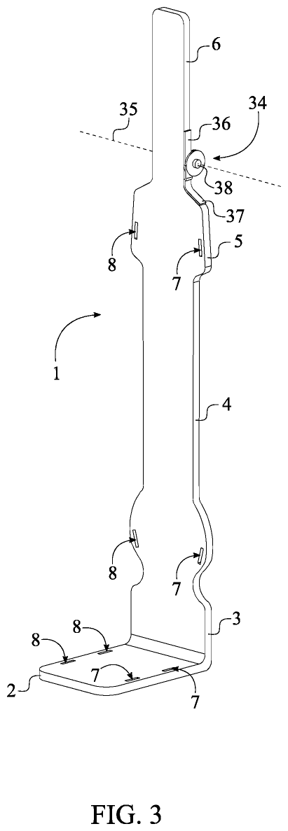

In reference to FIGS. 1 and 3. The present invention is a new crutch system with a versatile crutch that changes between a crutch and cane and other walking aids. The present invention saves the user a significant amount of time and effort, as the user does not need to visit medical facilities to customize the length of the versatile crutch. The present invention comprises a leg stabilizer 1, a plurality of stabilizer straps 9, a knee brace 10, and a walking-aid apparatus 15. The plurality of stabilizer straps 9 is made from nylon or other durable material, configured to secure the user's injured foot and leg to the leg stabilizer 1. The leg stabilizer 1 is an “L” shaped component with 90-degree angle. The leg stabilizer 1 is configured to keep the user's leg in a straight position and keep the user's foot and heel in a 90-degree angle to facilitate the mending of broken bones or other conditions. The leg stabilizer 1 comprises a plurality of medial strap openings 7 and a plurality of lateral strap openings 8. The plurality of medial strap openings 7 is positioned along a medial side of the user's leg and is configured to engaged with the plurality of stabilizer straps 9. The plurality of lateral strap openings 8 is positioned along a lateral side of the user's leg and is configured to engaged with the plurality of stabilizer straps 9. Each of the plurality of stabilizer straps 9 is looped around a corresponding medial strap openings from the plurality of medial strap openings 7 and a corresponding lateral strap openings from the plurality of lateral strap openings 8. In other words, the user introduce each of the plurality of stabilizer straps 9 through one of the plurality of medial strap openings 7 and loops each of the plurality of the stabilizer straps 9 around the front of the leg thus securing one end of each of the plurality of stabilizer straps 9. Similarly, the user inserts each of the plurality of stabilizer straps 9 through one of the plurality of lateral strap openings 8 and loops each of the plurality of the stabilizer straps 9 around the front of the leg thus securing the opposite end of each of the plurality of stabilizer straps 9.

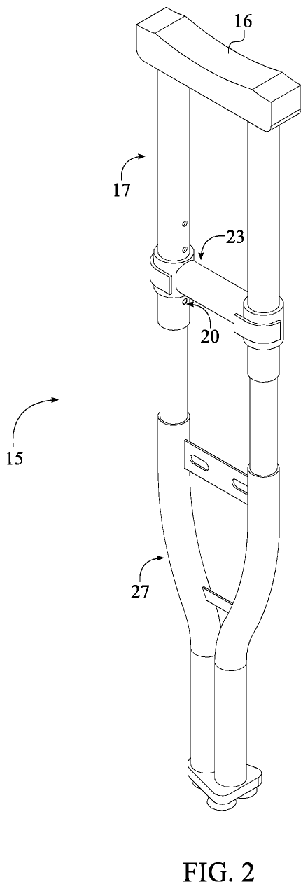

In reference to FIG. 2, the walking-aid apparatus 15 is a telescopic supporting device. The user may hold the walking-aid apparatus 15 under the armpit or in the hand to support the injured leg. The walking-aid apparatus 15 comprises a first handle 16, a telescopic upper-support assembly 17, a second handle 23, and a lower support 27. The first handle 16 is terminally connected to the telescopic upper-support assembly 17. The user may place the first handle 16 under the armpit or hold the handle in the hand to support the injured leg. The first handle 16 may have a layer of neoprene pad, which serve as a cushion when the user places the first handle 16 under the armpit. The second handle 23 is also connected to the telescopic upper-support assembly 17 and positioned offset of the first handle 16. When the user places the first handle 16 under the armpit to support the injured leg, the user may place the hand on the second handle 23 for further supporting the injured leg. The lower support 27 is adjacently connected to the telescopic upper-support assembly 17, opposite of the first handle 16. The lower support 27 converges the walking-aid apparatus 15 to reach a singular ground-contacting point.

In reference to FIGS. 1 and 3, the leg stabilizer 1 comprises an adjustable angle hinge 34, a foot plate 2, a heel plate 3, a calf plate 4, a knee plate 5, and a thigh plate 6. The foot plate 2 is perpendicularly positioned to the heel plate 3. The foot plate 2 is adjacently connected to the heel plate 3. The user may place the foot of the injured leg onto the foot plate 2 and place the heel of the injured leg against the heel plate 3 to keep the heel and the foot perpendicular to each other. The calf plate 4 is terminally connected to the heel plate 3, opposite to the foot plate 2. After the user put the heel of the injured leg against the heel plate 3, the user needs to ensure the entire back of the calf is fully contacting the calf plate 4 thus allowing the calf plate 4 to provide proper support to the user's calf. The knee plate 5 is terminally connected to the calf plate 4, opposite to the heel plate 3. The user may place the entire back of the knee against the knee plate 5 to keep knee joint straight. The thigh plate 6 is terminally connected to the knee plate 5, opposite to the calf plate 4 by the adjustable angle hinge 34. The user may adjust the angle between the thigh plate 6 and the knee plate 5 with the adjustable angel hinge 34, in case the user wishes to strap the injured leg to the leg stabilizer 1 in different angle. The user may put the back of the thigh to the thigh plate 6 to keep the entire leg, from thigh to the heel, totally straight as the user properly secure the entire leg to the leg stabilizer 1 with the plurality of stabilizer straps 9. The straight configuration of the leg stabilizer 1 is configured to keep the injured leg in a straight position to prevent extensor lag syndrome. However, the user may adjust the angle between the thigh plate 6 and the knee plate 5 by the adjustable angle hinge 34 to accommodate different injuries or circumstances.

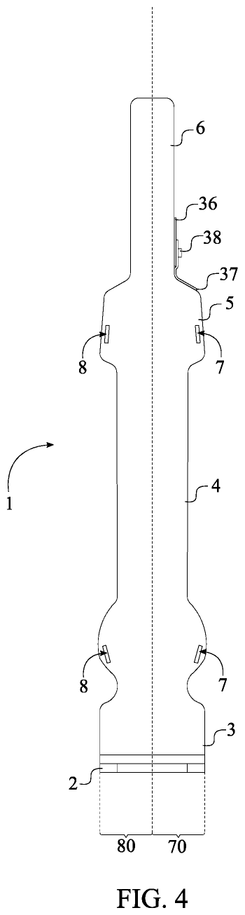

In reference to FIGS. 3 and 4, the adjustable angle hinge 34 comprises a rotational axis 35, a first arm 36, a second arm 37, and a hinge lock 38. The first arm 36 and the second arm 37 are rotatably engaged with each other around the rotational axis 35. The user may adjust the angle formed by the first arm 36 and the second arm 37 by rotate the first arm 36 or the second arm 37 to a different angle along the rotational axis 35. The first arm 36 is terminally integrated onto the thigh plate 6. The user may adjust the angular position of the thigh plate 6 relative to the knee plate 5 by changing the angle of the first arm 36 relative to the second arm 37. The second arm 37 is terminally integrated onto the knee plate 5. The user may adjust the angular position of the knee plate 5 relative to the thigh plate 6 by changing the angle of the second arm 37 relative to the first arm 36. The hinge lock 38 is integrated into the first arm 36 and the second arm 37. The hinge lock 38 is configured to allow the user to lock the first arm 36 and the second arm 37 in a certain angle. When the user straps the injured leg to the leg stabilizer 1 and adjust the first arm 36 and the second arm 37 to a proper angle, the user may lock the hinge lock 38 to maintain the same angle between the thigh plate 6 and the knee plate 5 thus preventing unwanted movement within the leg stabilizer 1.

In reference to FIGS. 1 and 4, the plurality of medial strap openings 7 traverses through a medial side 70 of the leg stabilizer 1. The plurality of medial strap openings 7 is positioned along the medial side 70 of the leg stabilizer 1. The plurality of medial strap openings 7 is configured to allow the user to introduce the plurality of stabilizer straps 9 through the leg stabilizer 1 to secure the user's leg to the medial side 70 of the leg stabilizer 1. The plurality of lateral strap openings 8 traverses through a lateral side 80 of the leg stabilizer 1. The plurality of lateral strap openings 8 is positioned along the lateral side 80 of the leg stabilizer 1. The plurality of lateral strap openings 8 is configured to allow the user to introduce the plurality of stabilizer straps 9 through the leg stabilizer 1 to secure the user's leg to the lateral side 80 of the leg stabilizer 1.

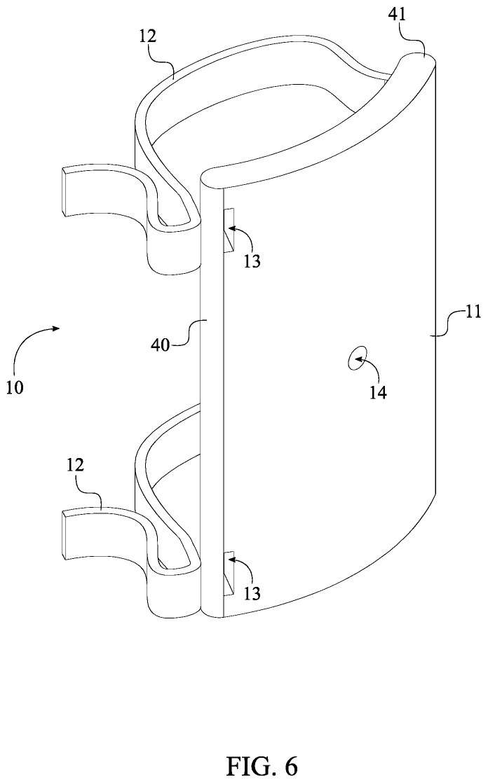

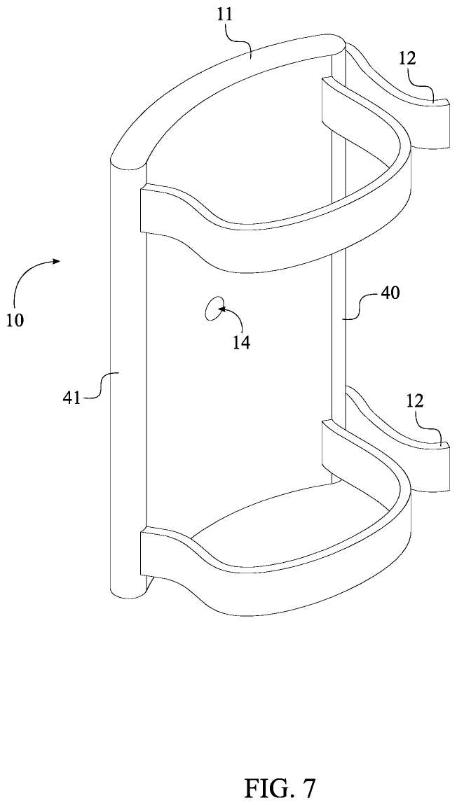

In reference to FIGS. 6 and 7, the knee brace 10 is configured to protect the front portion of the knee. The knee brace 10 comprises a brace body 11, a plurality of brace straps 12, a plurality of strap openings 13, and at least one ventilation hole 14. The plurality of strap openings 13 is linearly distributed along a first edge 40 of the brace body 11. The plurality of strap openings 13 is traversing through the brace body 11. The plurality of strap openings 13 is configured to allow the user to introduce each of the corresponding of the plurality of brace straps 12 through the plurality of strap openings 13 and secure the brace body 11 onto the front of the user's knee. The plurality of brace straps 12 is linearly distributed along a second edge 40 of the brace body 11, opposite to the plurality of strap openings 13. The plurality of brace straps 12 is connected onto the brace body 11. After the user places the knee brace 10 in front of the knee, the user may pull the free end of the plurality of brace straps 12 and wrap around the back of the user's knee, then introduce each of the plurality of brace straps 12 into each of the plurality of strap openings 13, prospectively, and secure the brace body 11 onto the front of the user's knee.

In reference to FIGS. 4, 6 and 7, in another embodiment, the knee brace 10 not only covers the front portion of the knee but also extends to both the lateral side and the medial side of the knee, furthermore, the present invention comprises at least one medial attachment clip and at least one lateral attachment clip. The at least one medial attachment clip and the at least one lateral attachment clip each comprises a first interlocking fastener and a second interlocking fastener. The first interlocking fastener is terminally connected to the brace body 11, and the second interlocking fastener is terminally connected to the knee plate 5. After the user properly secure the injured leg onto the leg stabilizer 1, the user may place the knee brace 10 on the top of the knee and attach the first interlocking fastener and the second interlocking fastener of the at least one medial attachment clip and at least one lateral attachment clip to each other, prospectively. The knee brace 10 now completely covers the front, medial, and lateral side of the knee, and form a complete cover of the entire knee with the leg stabilizer 1.

In reference to FIGS. 6 and 7, the at least one ventilation hole 14 is positioned between the plurality of strap openings 13 and the plurality of brace straps 12. This configuration is to prevent the user from introducing the plurality of brace straps 12 into the at least one ventilation hole 14. The at least one ventilation hole 14 is centrally traversing through the brace body 11. The at least one ventilation hole 14 is configured to improve the airflow for the user's knee to prevent the user's knee from overheating or sweating.

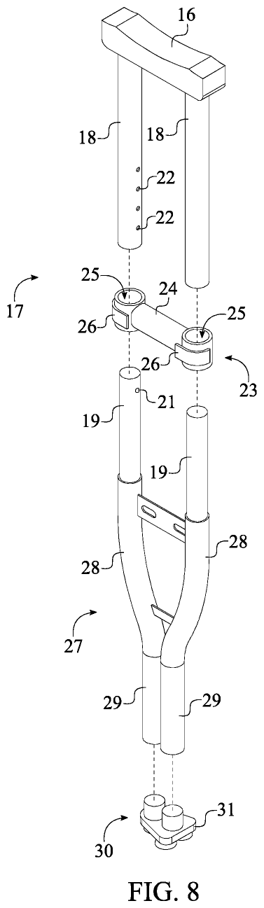

In reference to FIGS. 2 and 8, the telescopic upper-support assembly 17 comprises a pair of outer sleeves 18, a pair of inner sleeves 19, and at least one spring-loaded locking mechanism 20. The telescopic upper-support assembly 17 is configured to allow the user to modify the length of the walking-aid apparatus 15, as the height needs to be adjusted during different stages of the healing process. The spring-loaded mechanism comprises at least one spring plunger 21 and at least one opening 22. The pair of outer sleeves 18 is telescopically engaged around the pair of inner sleeves 19. The user may adjust the length of the walking-aid apparatus 15 by modifying the length of the pair of outer sleeves 18 around the pair of inner sleeves 19. The at least one spring plunger 21 is mounted within the pair of inner sleeves 19. The at least one opening 22 is traversing through the pair of outer sleeves 18. The at least one spring plunger 21 is selectively engaged with the at least one opening 22. The user may telescopically adjust the length of the telescopic upper-support assembly 17 by pressing down on the at least one spring plunger 21. The at least one spring plungers 21 is then retracted from the at least one opening 22 and also unlock the pair of outer sleeves 18 from the pair of inner sleeves 19. The user may now increase the length by pulling the pair of outer sleeves 18 away from the pair of inner sleeves 19 or reduce the length by inserting the pair of outer sleeves 18 into the pair of inner sleeves 19. When the walking-aid assembly is adjusted to an appropriate length, the at least one spring plungers 21 is released into the at least one opening 22, and the pair of outer sleeves 18 is then locked in the same location relative to the pair of inner sleeves 19.

The second handle 23 comprises a handle body 24, a pair of openings 25, and a latch-locking mechanism 26. The user may grip onto the handle body 24 for further support when placing the first handle 16 under the armpit. The pair of openings 25 is traversing through the handle body 24. This configuration allows the latch-locking mechanism 26 to lock the second handle 23 in place. The latch-locking mechanism 26 is integrated into the handle body 24, adjacent to the pair of openings 25. The latch-locking mechanism 26 is configured to allow the user to lock or unlock the second handle 23 with respect to the telescopic upper-support assembly 17. The pair of openings 25 is encircled around the pair of outer sleeves 18 of the telescopic upper-support assembly 17. After the latch-locking mechanism 26 is unlocked, the user may adjust the second handle 23 by moving the second handle 23 to different locations on the pair of outer sleeves 18 via the pair of openings 25.

In reference to FIGS. 1 and 8, the lower support 27 comprises a pair of bent sleeves 28, a pair of elongated sleeves 29, and a replaceable foot 30. The pair of bent sleeves 28 is adjacently connected to the pair of elongated sleeves 29. The pair of elongated sleeves 29 extends the convergence of the pair bent sleeves 28 and forms a bridge between the pair of bent sleeves 28 and the replaceable foot 30. The replaceable foot 30 is terminally attached to the pair of elongated sleeves 29, opposite to the pair of bent sleeves 28. The replaceable foot 30 is configured to contact with the ground as the user uses the walking-aid apparatus 15 to support the injured leg. The pair of bend sleeves 28 is terminally connected to the pair of inner sleeves 19 of the telescopic upper-support assembly 17, opposite of the pair of elongated sleeves 29. The pair of bent sleeves 28 is configured to converge and reducing the ground contacting point to one replaceable foot 30.

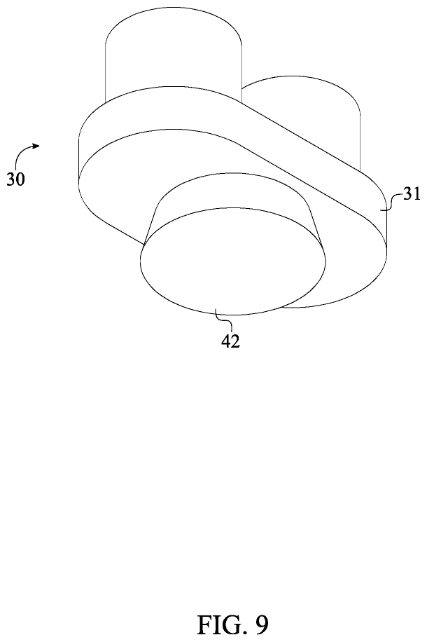

In some embodiment of the replaceable foot 30 comprises a toe body 31 and a first support 42 as shown in FIG. 9. The toe body 31 is attached to the pair of elongated sleeves 29. The toe body 31 is the bridging member between the first support 42 and the pair of elongated sleeves 29. The first support 42 is adjacently connected to the toe body 31, opposite of the pair of elongated sleeves 29. The first support 42 may be used by a user in the last stage of recovery because the first support 42 is lighter and provides less friction with the ground due to less surface area contacting the ground.

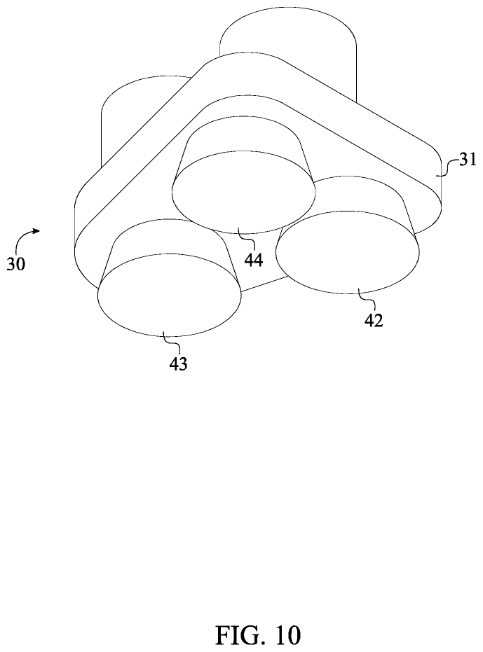

In some embodiment of the replaceable foot 30 comprises the toe body 31, the first support 42, a second support 43, and a third support 44 as shown in FIG. 10. The toe body 31 is attached to the pair of elongated sleeves 29. The toe body 31 is the bridging member between the first support 42, the second support 43, the third support 44, and the pair of elongated sleeves 29. The first support 42, the second support 43, and the third support 44 are perimetrically connected to the toe body 31, opposite of the pair of elongated sleeves 29. The replaceable foot 30 with the first support 42, the second support 43, and the third support 44 may be used by an older user, because this embodiment provides two additional contacting points with the ground, therefore it is more adaptive to different terrains than the replaceable foot 30 with only the first support 42.

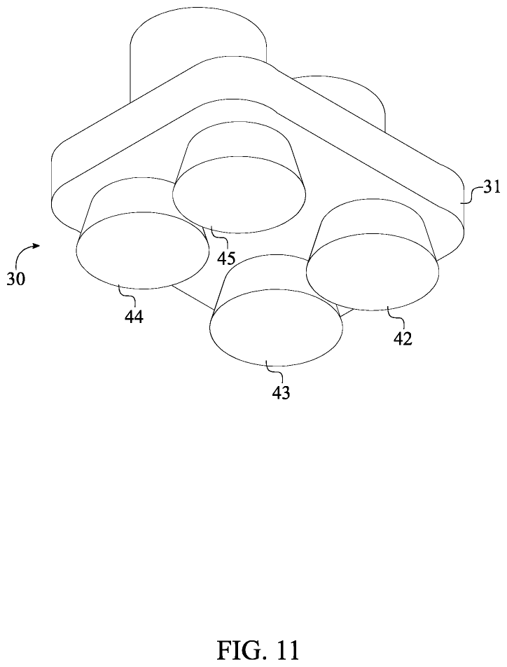

In some embodiment of the replaceable foot 30 comprises the toe body 31, the first support 42, the second support 43, the third support 44, and a fourth support 45 as shown in FIG. 11. The toe body 31 is attached to the pair of elongated sleeves 29. The toe body 31 is bridging between the first support 42, the second support 43, the third support 44, and the fourth support 45 and the pair of elongated sleeves 29. The first support 42, the second support 43, the third support 44, and the fourth support 45 are perimetrically connected to the toe body 31, opposite of the pair of elongated sleeves 29. The replaceable foot 30 with the first support 42, the second support 43, the third support 44, and the fourth support 45 may be used by older user, because this embodiment provides three additional contacting points with the ground, therefore it is more adaptive to different terrains than the replaceable foot 30 with only the first support 42.

Although the invention has been explained in relation to its preferred embodiment, it is to be understood that many other possible modifications and variations can be made without departing from the spirit and scope of the invention.

What is claimed is:

1. A crutch system comprises:
a leg stabilizer;
a plurality of stabilizer straps;
a knee brace;
a walking-aid apparatus;
the walking-aid apparatus comprising a first handle, a telescopic upper-support assembly, a second handle, and a lower support;
the leg stabilizer comprising a plurality of medial strap openings and a plurality of lateral strap openings;
each of the plurality of stabilizer straps being looped around a corresponding medial strap opening from the plurality of medial strap openings and a corresponding lateral strap opening from the plurality of lateral strap openings;
the first handle being terminally connected to the telescopic upper-support assembly;
the second handle being connected to the telescopic upper-support assembly;
the second handle being positioned offset of the first handle; and
the lower support being adjacently connected to the telescopic upper-support assembly, opposite of the first handle.

2. The crutch system as claimed in claim 1 comprises:
an adjustable angle hinge;
the leg stabilizer comprising a foot plate, a heel plate, a calf plate, a knee plate, and a thigh plate;
the foot plate being perpendicularly positioned to the heel plate;
the foot plate being adjacently connected to the heel plate;
the calf plate being terminally connected to the heel plate, opposite to the foot plate;
the knee plate being terminally connected to calf plate, opposite to the heel plate; and
the thigh plate being terminally connected to the knee plate by the adjustable angle hinge, opposite to the calf plate.

3. The crutch system as claimed in claim 2 comprises:
the adjustable angle hinge comprising a rotational axis, a first arm, a second arm, and a hinge lock;
the first arm and the second arm being rotatably engaged with each other around the rotational axis;
the first arm being terminally integrated into the thigh plate;
the second arm being terminally integrated into the knee plate; and
the hinge lock being integrated into the first arm and the second arm.

4. The crutch system as claimed in claim 1, wherein the plurality of medial strap openings traversing through a medial side of the leg stabilizer.

5. The crutch system as claimed in claim 1, wherein the plurality of lateral strap openings traversing through a lateral side of the leg stabilizer.

6. The crutch system as claimed in claim 1 comprises:
the knee brace comprising a brace body, a plurality of brace straps, a plurality of strap openings, and at least one ventilation hole;
the plurality of strap openings being linearly distributed along a first edge of the brace body;
the plurality of strap openings traversing through the brace body;
the plurality of brace straps being linearly distributed along a second edge of the brace body, opposite to the plurality of strap openings;
the plurality of brace straps being connected onto the brace body;
the at least one ventilation hole being positioned between the plurality of strap openings and the plurality of brace straps; and
the at least one ventilation hole centrally traversing through the brace body.

7. The crutch system as claimed in claim 1 comprises:
the telescopic upper-support assembly comprising a pair of outer sleeves, a pair of inner sleeves and at least one spring-loaded locking mechanism;
the spring-loaded locking mechanism comprising a plurality of spring plungers and at least one opening;
the pair of outer sleeves being telescopically engaged around the pair of inner sleeves;
the plurality of spring plungers being mounted within the pair of inner sleeves;
the at least one opening traversing through the pair of outer sleeves; and
the plurality of spring plungers being selectively engaged with the at least one opening.

8. The crutch system as claimed in claim 1 comprises:
the second handle comprising a handle body, a pair of openings, and a latch-locking mechanism;
the pair of openings traversing through the handle body;
the latch-locking mechanism being integrated into the handle body, adjacent to the pair of openings; and
the pair of openings being encircled around a pair of outer sleeves of the telescopic upper-support assembly.

9. The crutch system as claimed in claim 1 comprises:
the lower support comprises a pair of bent sleeves, a pair of elongated sleeves, and a replaceable foot;
the pair of bent sleeves being adjacently connected to the pair of elongated sleeves;
the replaceable foot being terminally attached to the pair of elongated sleeves, opposite to the pair of bent sleeves; and
the pair of bend sleeves being terminally connected to a pair of inner sleeves of the telescopic upper-support assembly, opposite of the pair of elongated sleeves.

10. The crutch system as claimed in claim 9 comprises:
the replaceable foot comprises a toe body and a first support;
the toe body being attached to the pair of elongated sleeves; and
the first support being adjacently connected to the toe body, opposite of the pair of elongated sleeves.

11. The crutch system as claimed in claim 9 comprises:
the replaceable foot 30 comprises a toe body, a first support, a second support, and a third support;
the toe body being attached to the pair of elongated sleeves; and
the first support, the second support, and the third support being perimetrically connected to the toe body, opposite of the pair of elongated sleeves.

12. The crutch system as claimed in claim 9 comprises:
the replaceable foot comprises a toe body, a first support, a second support, a third support, and a fourth support;
the toe body being attached to the pair of elongated sleeves; and
the first support, the second support, the third support, and the fourth support being perimetrically connected to the toe body, opposite of the pair of elongated sleeves.
